(19) United States
(12) Patent Application Publication
(10) Pub. No.: US 2011/0008308 A1
(43) Pub. Date: Jan. 13, 2011

(54) Composition for hair, skin, nails

(76) Inventors: Robert Peter Taylor, London (GB); Kartar Singh Lalvani, London (GB)

Correspondence Address: Robert Taylor;Vitabiotics Limited, 1 Apsley Way, London, NW2 7HF, GB

(21) Appl. No.: 12/804,192

(22) Filed: Jul. 16, 2010

(30) Foreign Application Priority Data
Mar. 6, 2009 (GB) PCT/GB2009/000621

(51) Int. Cl. A61K 38/43; A61P 17/00; A61P 3/02; A61P 39/06

(52) U.S. Cl. 424/94.1

(57) ABSTRACT

A pharmaceutical composition for use in hair, skin and nail health maintenance, for combined, sequential or simultaneous administration, in any form, via any biological route. In its optimal embodiment the composition consists of a daily intake of 1 tablet that consists of: 5 mcg Vitamin D3, 40 mg Vitamin E (natural source), 60 mg Vitamin C, 8 mg Thiamin (vitamin B1), 4 mg Riboflavin (vitamin B2), 18 mg Niacin (vitamin B3), 10 mg Vitamin B6, 500 mcg Folic Acid, 9 mcg Vitamin B12, 45 mcg Biotin, 40 mg Pantothenic Acid, 12 mg Iron, 75 mg Magnesium, 15 mg Zinc, 200 mcg Iodine, 0.5 mg Manganese, 1 mg Copper, 50 mcg Chromium, 100 mcg Selenium, 10 mg L-Cystine, 2 mg Natural Mixed Carotenoids, 15 mg Grape Seed Extract (95% proanthocyanidins); plus 1 Nutri-dermal softgel capsule that consists of: 300 mg Omega-3 Fish Oil, 6 mg Lutein, 200 mg Starflower Oil, 50 mg Blackcurrant Seed Oil, 4 mg Lycopene, and 5 mg Coenzyme Q10.

### BACKGROUND OF THE INVENTION

This invention concerns a composition of biochemical or cosmeceutical constituents for use in the maintenance of the health of skin, hair and nails.

We are living in an image conscious society where spending on beauty and beauty regimes has never been higher. In recent years this has extended into the supplement area as more people are aware of the benefits of seeking “beauty from within” and are increasingly adopting higher price “cosmeceuticals”. There is an increasing interest in not only maintaining the health of the hair and nails, but also a particular interest in maintaining the health of their skin.

Beauty supplement sales have grown by 5% in the last two years and this growth trend is expected to escalate with a market growth between 10% and 30% in the next 5 years. Therefore, beauty supplements can no longer be considered a niche market as more consumers become convinced of the benefits of seeking beauty through nutrition. Research showed that almost a third of the U.K. population, made up primarily of women and young girls, had used beauty supplements or would consider using them in the future. Most contain vitamins, antioxidants and omega-3 fatty acids.

Those in primary need are women between the ages of 35 and 45 years old who would like to protect against the visible signs of skin ageing. Also in primary need are women between 18 and 35 years old who are not yet concerned about the signs of ageing but who want to have healthy skin, hair, and nails in order to look good when socialising.

Those in secondary need are women of any age who have specific “problem” areas to address, most notably their skin, e.g. blemishes, redness, oily skin.

### BRIEF SUMMARY OF THE INVENTION

This invention concerns a formulation aimed at supplementing the diet in order to provide constituents that will optimise the health of the hair, skin and nails. It has been developed for those individuals who while they are concerned about maintaining the health of their hair and nails have a particular interest in maintaining the health of their skin.

According to this invention, there is a pharmaceutical composition, or the use of that composition in the manufacture of a medicament, or a method of tre including the use of that pharmaceutical composition for combined, sequential or simultaneous administration, in any form, via any route, for use in the maintenance of skin and/or nail and/or hair health. In its optimal form it consists of a daily intake of 1 tablet that consists of: 5 mcg Vitamin D3, 40 mg Vitamin E (natural source), 60 mg Vitamin C, 8 mg Thiamin (vitamin B1), 4 mg Riboflavin (vitamin B2), 18 mg Niacin (vitamin B3), 10 mg Vitamin B6, 500 mcg Folic Acid, 9 mcg Vitamin B12, 45 mcg Biotin, 40 mg Pantothenic Acid, 12 mg Iron, 75 mg Magnesium, 15 mg Zinc, 200 mcg Iodine, 0.5 mg Manganese, 1 mg Copper, 50 mcg Chromium, 100 mcg Selenium, 10 mg L-Cystine, 2 mg Natural Mixed Carotenoids, 15 mg Grape Seed Extract (95% proanthocyanidins); plus 1 Nutri-dermal softgel capsule that consists of: 300 mg Omega-3 Fish Oil, 6 mg Lutein, 200 mg Starflower Oil, 50 mg Blackcurrant Seed Oil, 4 mg Lycopene, and 5 mg Coenzyme Q10.

### DETAILED DESCRIPTION OF THE INVENTION

The inclusion of a nutri-dermal capsule provides essential Omega-3 and 6 fatty acids, which can help maintain a healthy looking complexion. It also includes a novel combination ingredients including lycopene; lutein and co-enzyme Q10 which help protect skin cells from the damaging effects of free radicals propagated by UV light and pollution.

The following is the optimal formulation—

Daily dosages of the following constituents, provided in 1 tablet per day:
Vitamin D3—5 mcg
Vitamin E (natural source)—40 mg
Vitamin C—60 mg
Thiamin (vitamin B1)—8 mg
Riboflavin (vitamin B2)—4 mg
Niacin (vitamin B3)—18 mg
Vitamin B6—10 mg
Folic Acid—500 mcg
Vitamin B12—9 mcg
Biotin—45 mcg
Pantothenic Acid—40 mg
Iron—12 mg
Magnesium—75 mg
Zinc—15 mg
Iodine—200 mcg
Manganese—0.5 mg
Copper—1 mg
Chromium—50 mcg
Selenium—100 mcg
L-Cystine—10 mg
Natural Mixed Carotenoids—2 mg
Grape Seed Extract (95% proanthocyanidins)—15 mg

Plus, daily dosages of the following constituents, provided in 1 Nutri-dermal softgel capsule per day:
Omega-3 Fish Oil—300 mg
Lutein—6 mg
Starflower Oil—200 mg
Blackcurrant Seed Oil—50 mg
Lycopene—4 mg
Co-Q10—5 mg

Starflower oil and Omega-3 Fish Oil constitute the majority of the composition.

In more concise versions of the composition, Starflower oil and Omega-3 Fish Oil constitute the majority of the composition, and are included with Proanthocyanidins, Coenzyme Q10, and optionally Lycopene, and one or two or more of: Vitamin D3, Vitamin E, Vitamin C, Thiamin (vitamin B1), Riboflavin (vitamin B2), Niacin (vitamin B3), Grape Seed Extract, Vitamin B6, Folic Acid, Vitamin B12, Biotin, Pantothenic Acid, Iron, Magnesium, Zinc, Iodine, Manganese, Copper, Chromium, Selenium, L-Cystine, Natural Mixed Carotenoids, Lutein, Blackcurrant Seed Oil.

### RDA/Active Levels:

The following are the recommended dietary allowances for some of the constituents, and the reasons for the level chosen:
Vitamin D3 (200 iu)—100%—Exact RDA level as a nutritional safeguard.
Natural Vitamin E—400%—Higher than RDA for increased antioxidant protection. Vitamin E is known to help heal the skin.
Vitamin C—100%—Exact RDA level as a nutritional safeguard and important as a cofactor for the production of collagen.
Vitamin B1—571%—Because vitamin B1 is a water-soluble vitamin, any excess is excreted by the body making it safe to take at relatively high doses. Every cell of the body requires Vitamin B1.
Vitamin B2—250%—Because vitamin B2 is a water-soluble vitamin, the body excretes any excess. It is also of benefit to those who have cut red meat from their diet. A long-term intake of up to 200 mg daily is not considered harmful.
Vitamin B3 (Niacin)—100%—Because Niacin is a water-soluble vitamin, the body excretes any excess. It is also of benefit to those who have cut red meat from their diet. A long-term intake of up to 500 mg daily is not considered harmful.
Vitamin B6—500%—Because vitamin B6 is a water-soluble vitamin, the body excretes any excess. A long-term intake of up to 200 mg daily is not considered harmful.
Folic Acid—250%—To safeguard women who are pregnant or planning to become pregnant who are advised to take a daily supplement containing 400 mcg of folic acid, to help prevent neural tube defects in the developing baby such as spina bifida. Folic acid is also involved in red blood cell production.
Vitamin B12—900%—Because vitamin B12 is a water-soluble vitamin, any excess is excreted by the body making it safe to take at relatively high doses. A long-term intake of up to 3000 mcg daily is not considered harmful.
Biotin—30%—Widely distributed by many foods and also synthesised by the body.
Pantothenic Acid—667%—Most people need to have Pantothenic Acid supplementation due to changing lifestyles. Primitive human diets provided greater amounts of this nutrient than is found in modern diets.
Iron—86%—Excess iron in the tissues may lead to the production of free radicals and increase the need for vitamin E.
Magnesium—25%—Below RDA level to take account of other dietary/supplement sources.
Zinc—100%—RDA level.
Iodine—133%—A lack of thyroxine hormone has a detrimental effect on the appearance of skin, hair and nails.
Manganese—No RDA level established
Copper—No RDA level established
Chromium GTF—No RDA level established
Selenium—No RDA level established
Cystine—No RDA level established
Natural Mixed Carotenoids—No RDA level established
Grape Seed Extract (95% proanthocyanidins)—No RDA level established
Omega-3 Fish Oil—No RDA level established
Lutein—No RDA level established
Starflower Oil—No RDA level established
Blackcurrant Seed Oil—No RDA level established
Lycopene—No RDA level established
Co-Q10—No RDA level.

### Relevance of Constituents

The composition provides a significant dietary level of nutrients and ensures that supplemental intake is within safety levels to help maintain healthy, skin, hair and nails. It provides particular support for skin. It also provides essential nutritional building blocks and protective factors for skin, hair and nails. The nutri-dermal capsule contains specialist ingredients including essential fatty acids for skin condition and additional antioxidants to help protect skin from free radical damage. One tablet will provide the ideal dietary level of nutrients and ensures that supplemental intake is within safety levels. One tablet a day will also ensure the skin, hair and nails receive all the nutrients they need from within. One nutri-dermal capsule provides a selection of nutrients specifically targeted at skin health.

Constituents are included for the following reasons:

Vitamin D3 plays a role in skin cell metabolism and growth and may be especially helpful for people with dry skin and psoriasis.

Vitamin E (natural source) scavenges free radicals, helping to protect against the ageing of skin tissues, which can cause wrinkles. It is also useful for stretch marks, scar tissue, and to reduce inflammation caused by sunburn.

Vitamin C is a central antioxidant and is also a cofactor in the production of collagen, which gives skin strength and elasticity. Hangnails and inflammation of the tissue surrounding the nail (paronychia) are linked to vitamin C deficiency.

The B complex vitamins: Thiamin (vitamin B1), Riboflavin (vitamin B2), Niacin (vitamin B3),Vitamin B6, Folic Acid, Vitamin B12, are involved in red blood cell production and blood circulation, thereby increasing the transport of nutrients to the dermis.

Biotin stimulates growth of body cells and related to hair growth.

Pantothenic Acid is essential in the metabolism of carbohydrate, fat & protein, for energy release.

Iron deficiency can produce “spoon” nails and/or vertical ridges and can cause nails to become thin.

Magnesium deficiency can lead to hair loss.

Zinc is needed for the formation of new nail material in the nail bed. Brittleness and white spots on nails are a common sign of zinc deficiency.

Iodine is essential for the production of thyroxine, a hormone which regulates metabolic rate. A lack of thyroxine can have a detrimental effect on the appearance of skin, hair and nails.

Manganese is vital for healthy skin because of its role in connective tissue metabolism.

Copper is necessary for hair strength and formation of hair follicles.

Chromium plays a key role in fat and carbohydrate metabolism providing energy for skin cells. Plays a role in the body's glucose tolerance factor, important for individuals with a poor glucose balance which can in turn effect skin coloration and health.

Selenium is needed for antioxidants that help protect against skin damage caused by free radicals, UV light and pollution.

L-Cystine, the sulphur containing amino acid is necessary for skin and nail growth.

Natural Mixed Carotenoids helps protect skin from harmful effects of UV light.

Grape Seed Extract (95% proanthocyanidins) contains antioxidants to help protect cells from free radical damage and promote a healthy circulation.

Lutein helps protect skin from free radical damage particularly that induced by UV radiation.

Omega-3 Fish Oil helps cells repair and prevent damage. It also helps with the risk of skin cancer, help the body resist sunburn, and help reduce fine lines, wrinkles, and ease dry skin.

Blackcurrant Seed Oil provides the plant world's richest known natural source of Omega-3 and Omega-6. These important fatty acids may help to keep skin supple and toned.

Lycopene can play a valuable role in protecting vulnerable skin from the sun's UV radiation, which can contribute to premature skin damage.

Co-Q10 as well as being an antioxidant it has been shown to decrease the activity of certain enzymes that degrade collagen present in skin.

### Suggested Daily Intake/Dosage:

It is best to take 1 tablet and 1 capsule per day with a main meal, with a full glass of water or cold drink.

### Recommended Contra-Indications/Warnings:

The tablet and capsule should only be taken on a full stomach. This is recommended to ensure maximum absorption of nutrients and to avoid nausea which can occur when taking any tablets or capsules without food

No additional multivitamin is required.

1. A pharmaceutical composition for use in hair, skin and nail health maintenance, for combined, sequential or simultaneous administration, in any form, via any biological route, the majority of which consists of Starflower Oil, Omega-3 Fish Oil, and Blackcurrant Seed Oil, and which also consists of: Vitamin D3, Vitamin E, Vitamin C, Thiamin (vitamin B1), Riboflavin (vitamin B2), Niacin (vitamin B3), Vitamin B6, Folic Acid, Vitamin B12, Biotin, Pantothenic Acid, Iron, Magnesium, Zinc, Iodine, Manganese, Copper, Chromium, Selenium, L-Cystine, Natural Mixed Carotenoids, Grape Seed Extract (95% proanthocyanidins), Lutein, Lycopene, Coenzyme Q10.

2. A claim according to claim 1, in which the average daily dosages of those substances other than Starflower Oil, Omega-3 Fish Oil, and Blackcurrant Seed Oil are: 5 mcg Vitamin D3, 40 mg Vitamin E, 60 mg Vitamin C, 8 mg Thiamin (vitamin B1), 4 mg Riboflavin (vitamin B2), 18 mg Niacin (vitamin B3), 10 mg Vitamin B6, 500 mcg Folic Acid, 9 mcg Vitamin B12, 45 mcg Biotin, 40 mg Pantothenic Acid, 12 mg Iron, 75 mg Magnesium, 15 mg Zinc, 200 mcg Iodine, 0.5 mg Manganese, 1 mg Copper, 50 mcg Chromium, 100 mcg Selenium, 10 mg L-Cystine, 2 mg Natural Mixed Carotenoids, 15 mg Grape Seed Extract (95% proanthocyanidins), 6 mg Lutein, 4 mg Lycopene, 5 mg Coenzyme Q10.

3. A claim according to claim 1 in which the range of average daily dosages are: 2.5 mcg to 7.5 mcg Vitamin D3, 20 mg to 60 mg Vitamin E, 30 mg to 90 mg Vitamin C, 4 mg to 12 mg Thiamin (vitamin B1), 2 mg to 6 mg Riboflavin (vitamin B2), 9 mg to 27 mg Niacin (vitamin B3), 5 mg to 15 mg Vitamin B6, 250 mcg to 750 mcg Folic Acid, 5 mcg to 15 mcg Vitamin B12, 25 mcg to 75 mcg Biotin, 20 mg to 60 mg Pantothenic Acid, 6 mg to 18 mg Iron, 25 mg to 75 mg Magnesium, 5 mg to 25 mg Zinc, 100 mcg to 300 mcg Iodine, 0.25 mg to 0.75 mg Manganese, 0.5 mg to 1.5 mg Copper, 25 mcg to 75 mcg Chromium, 50 mcg to 150 mcg Selenium, 5 mg to 15 mg L-Cystine, 1 mg to 3 mg Natural Mixed Carotenoids, 5 mg to 25 mg Grape Seed Extract (95% proanthocyanidins), 150 mg to 1000 mg Omega-3 fish oil, 25 mg to 75 mg Blackcurrant Seed Oil, 3 mg to 9 mg Lutein, 100 mg to 300 mg Starflower Oil, 2 mg to 6 mg Lycopene, 2 mg to 10 mg Coenzyme Q10.

4. A pharmaceutical composition for use in hair, skin and nail health maintenance, for combined, sequential or simultaneous administration, in any form, via any biological route, the majority of which consists of Starflower Oil, Omega-3 Fish Oil, and Blackcurrant Seed Oil, and which also comprises Proanthocyanidins, Coenzyme Q10, Lycopene, and 2 or more of: Vitamin D3, Vitamin E, Vitamin C, Thiamin (vitamin B1), Riboflavin (vitamin B2), Niacin (vitamin B3), Grape Seed Extract, Vitamin B6, Folic Acid, Vitamin B12, Biotin, Pantothenic Acid, Iron, Magnesium, Zinc, Iodine, Manganese, Copper, Chromium, Selenium, L-Cystine, Natural Mixed Carotenoids, Lutein.

5. A pharmaceutical composition for use in hair, skin and nail health maintenance, for combined, sequential or simultaneous administration, in any form, via any biological route, the majority of which consists of Starflower Oil, Omega-3 Fish Oil, and Blackcurrant Seed Oil, and which also comprises Proanthocyanidins, Coenzyme Q10, Lycopene, and one or more of: Vitamin D3, Vitamin E, Vitamin C, Thiamin (vitamin B1), Riboflavin (vitamin B2), Niacin (vitamin B3), Grape Seed Extract, Vitamin B6, Folic Acid, Vitamin B12, Biotin, Pantothenic Acid, Iron, Magnesium, Zinc, Iodine, Manganese, Copper, Chromium, Selenium, L-Cystine, Natural Mixed Carotenoids, Lutein.

6. A claim according to any one of claims 1 to 5 in which the pharmaceutical composition does not comprise more than 300 mg of amino acids in a protein or amino acid complex form.

7. A claim according to any one of claims 1 to 5 in which Vitamin D is used instead of Vitamin D3.

8. A claim according to any one of claims 1 to 5 in which DHA (Docosahexaenpoic acid) and EPA (Eicosapentaenoic acid) are used instead of Omega-3 fish oil.

9. A pharmaceutical composition according to any one of claims 1 to 5 for any use in hair or skin or nail health maintenance.

10. A claim according to any one of claims 1 to 5 in which the composition includes one or more carriers or excipients.

11. The use in the manufacture of a medicament using a composition as described in claims 1 to 10.
